(19) United States
(12) Patent Application Publication
(10) Pub. No.: US 2020/0037865 A1
(43) Pub. Date: Feb. 6, 2020

(54) IMAGE PROCESSING DEVICE, IMAGE PROCESSING SYSTEM, AND IMAGE PROCESSING METHOD

(71) Applicant: OLYMPUS CORPORATION, Tokyo (JP)

(72) Inventors: Kyosuke MIZUNO, Tokyo (JP); Ryuichi YAMAZAKI, Tokyo (JP); Kazuhiko HINO, Tokyo (JP); Takeshi SUGA, Tokyo (JP)

(73) Assignee: OLYMPUS CORPORATION, Tokyo (JP)

(21) Appl. No.: 16/596,977

(22) Filed: Oct. 9, 2019

Related U.S. Application Data
(63) Continuation of application No. PCT/JP2018/014720, filed on Apr. 6, 2018.

(30) Foreign Application Priority Data
Jun. 30, 2017 (JP) 2017-129630

(51) Int. Cl. A61B 1/045; A61B 1/00; A61B 1/06; H04N 13/254; H04N 13/324; H04N 13/359; G02B 23/24; H04N 5/225

(52) U.S. Cl. CPC A61B 1/045; A61B 1/00009; A61B 1/0661; H04N 13/254; H04N 2005/2255; H04N 13/359; G02B 23/2415; H04N 5/225; H04N 13/324

(57) ABSTRACT

An image processing device is configured to obtain first-type image data and second-type image data generated as a result of taking images of same photographic subject from different directions, and generate a parallax image based on the first-type image data and the second-type image data. The image processing device includes a processor comprising hardware. The processor is configured to: set an image selection mode in which an image is selectable based on an image displayed in a display; superimpose a frame data onto image data to be displayed in the display when the image selection mode is set, the frame data representing a boundary area whose size is set based on an optical system that forms an image of the photographic subject; and obtain the first-type image data and the second-type image data corresponding to selected image when the image selection mode is set.

### CROSS-REFERENCE TO RELATED APPLICATION

This application is a continuation of PCT International Application No. PCT/JP2018/014720 filed on Apr. 6, 2018, which designates the United States, incorporated herein by reference, and which claims the benefit of priority from Japanese Patent Application No. 2017-129630, filed on Jun. 30, 2017, incorporated herein by reference.

### BACKGROUND

### 1. Technical Field

The present disclosure relates to an image processing device, an image processing system, and an image processing method.

### 2. Related Art

In recent years, in the medical field, with the aim of facilitating diagnosis and examination, there is a demand for enabling medical observation of the observation target using stereoscopic images. In regard to such a demand, there is a known technology in which a parallax image is generated from two sets of image data, namely, image data for the left eye and image data for the right eye having mutual parallax, and displaying the parallax image as a stereoscopic image (for example, refer to Japanese Laid-open Patent Publication No. 2015-220643). If the technology disclosed in Japanese Laid-open Patent Publication No. 2015-220643 is implemented in an endoscope system that includes an endoscope and a processor, then the endoscope can obtain the image data for the left eye and the image data for the right eye, and the processor can generate a parallax image from the two sets of image data and stereoscopically display the image of the inside of the subject. In such a case, the endoscope has the following components installed therein: a left-eye optical system that forms an observation image for the left eye; a left-eye imaging device that receives the light obtained as a result of image formation performed by the left-eye optical system and generates image data; a right-eye optical system that forms an observation image for the right eye; and a right-eye imaging device that receives the light obtained as a result of image formation performed by the right-eye optical system and generates image data.

### SUMMARY

In some embodiments, provided is an image processing device configured to obtain first-type image data and second-type image data generated as a result of taking images of same photographic subject from different directions, and generate a parallax image based on the first-type image data and the second-type image data. The image processing device includes: a processor comprising hardware, wherein the processor is configured to: set an image selection mode in which an image is selectable based on an image displayed in a display; superimpose a frame data onto image data to be displayed in the display when the image selection mode is set, the frame data representing a boundary area whose size is set based on an optical system that forms an image of the photographic subject; and obtain the first-type image data and the second-type image data corresponding to selected image when the image selection mode is set.

In some embodiments, an image processing system includes: an imaging unit including an optical system configured to form a photographic subject image and an image sensor configured to receive light obtained as a result of image formation performed by the optical system and perform photoelectric conversion, the imaging unit being configured to generate first-type image data and second-type image data generated as a result of taking images of same photographic subject from different directions; and a processor comprising hardware. The processor is configured to set an image selection mode in which an image is selectable based on an image displayed in a display, superimpose a frame data onto image data to be displayed in the display when the image selection mode is set, the frame data representing a boundary area whose size is set based on the optical system that forms an image of the photographic subject, and obtain the first-type image data and the second-type image data corresponding to selected image when the image selection mode is set.

In some embodiments, provided is an image processing method of obtaining first-type image data and second-type image data generated as a result of taking images of same photographic subject from different directions, and generating a parallax image based on the first-type image data and the second-type image data. The image processing method includes: superimpose a frame data onto image data to be displayed in a display when observation mode is set to an image selection mode in which an image is selectable based on an image displayed in the display, the frame data representing a boundary area whose size is set based on an optical system that forms an image of the photographic subject; and obtaining the first-type image data and the second-type image data corresponding to selected image when the observation mode is set to the image selection mode.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an overall configuration of an endoscope system according to an embodiment of the disclosure;

FIG. 2 is a block diagram illustrating the overall configuration of the endoscope system according to the embodiment of the disclosure;

FIG. 3 is a block diagram for explaining a configuration of a signal processing unit of the endoscope system according to the embodiment of the disclosure;

FIG. 4 is a diagram illustrating an exemplary image displayed in a display device of the endoscope system according to the embodiment of the disclosure;

FIG. 5 is a diagram for explaining a parallax image generated in the endoscope system according to the embodiment of the disclosure; and

FIG. 6 is a flowchart for explaining the image processing performed in the endoscope system according to the embodiment of the disclosure.

### DETAILED DESCRIPTION

An illustrative embodiment (hereinafter, called “embodiment”) of the disclosure is described below. In the embodiment, as an example of an image processing system that includes an image processing device according to the disclosure, the explanation is given about a medical endoscope system that takes images of the inside of the subject such as a patient and displays the images. However, the disclosure is not limited by the embodiment. Moreover, with reference to the drawings, the same constituent elements are referred to by the same reference numerals.

### Embodiment

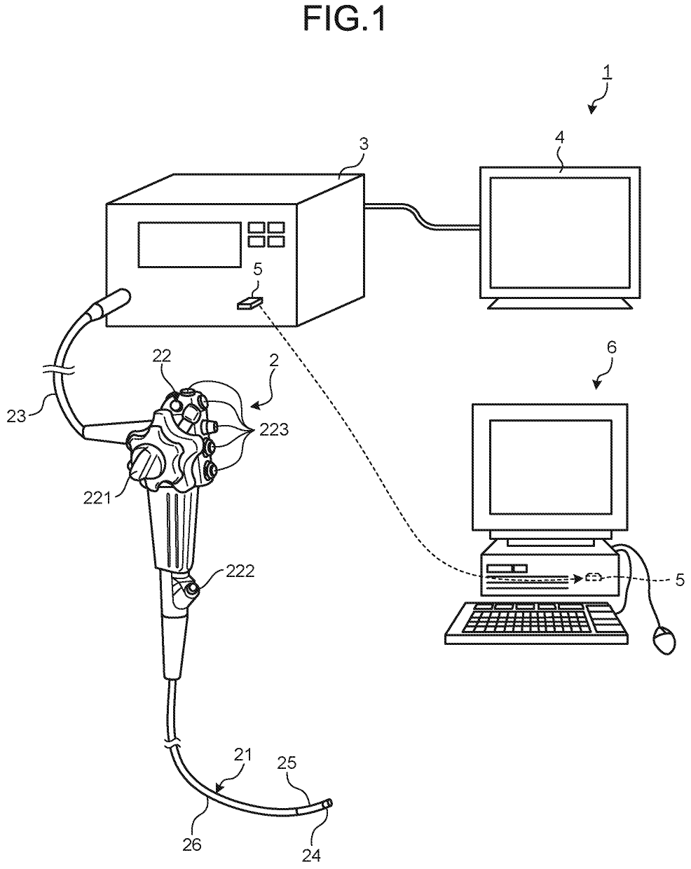

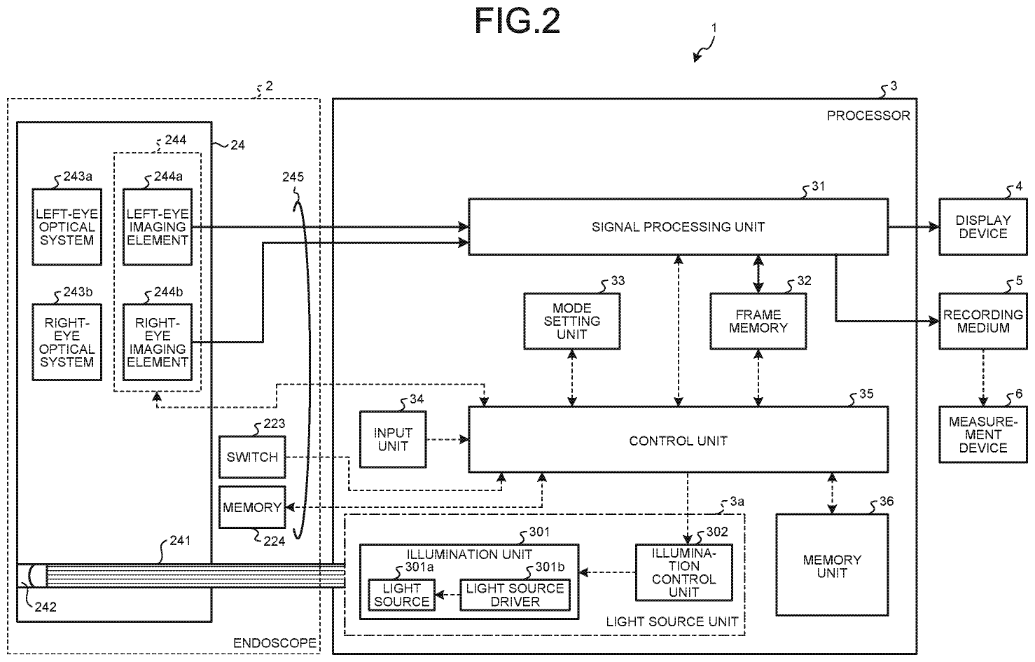

FIG. 1 is a diagram illustrating an overall configuration of the endoscope system according to the embodiment of the disclosure. FIG. 2 is a block diagram illustrating the overall configuration of the endoscope system according to the embodiment of the disclosure.

An endoscope system 1 illustrated in FIGS. 1 and 2 includes an endoscope 2 that, when the front end thereof is inserted inside the subject, takes in-vivo images of the subject; and includes a light source unit 3a that generates illumination light to be emitted from the front end of the endoscope 2. Moreover, the endoscope system 1 includes a processor 3 that performs predetermined signal processing with respect to imaging signals obtained by the endoscope 2 by performing imaging and that comprehensively controls the overall operations of the endoscope system 1; and a display device 4 that displays the in-vivo images generated as a result of the signal processing performed by the processor 3. To the processor 3 is electrically connected a recording medium 5 in which, for example, the data related to the images taken by the endoscope 2 can be recorded. The recording medium 5 is connected in a detachably-attachable manner to a measurement device 6 that is a separate device from the processor 3 and that is made to read the recorded data. The recording medium 5 is configured by connecting a USB memory to the processor 3 in a detachably-attachable manner. Meanwhile, in FIG. 2, solid arrows indicate transmission of electrical signals related to images, and dotted arrows indicate transmission of electrical signals related to the control.

The endoscope 2 includes the following: an insertion portion 21 that is flexible and has an elongated shape; an operating unit 22 that is connected to the proximal end of the insertion portion 21 and that receives input of various operation signals; and a universal cord 23 that extends from the operating unit 22 in a different direction than the direction of extension of the insertion portion 21 and that has various cables built-in for establishing connection with the processor 3 (including the light source unit 3a).

The insertion portion 21 includes the following: a front end portion 24 that has an imaging unit 244 built-in, in which pixels for generating signals by receiving light and performing photoelectric conversion are arranged in a two-dimensional manner; a freely-bendable curved portion 25 that is made of a plurality of bent pieces; and a flexible tube 26 that is a flexible and long tube connected to the proximal end of the curved portion 25. The insertion portion 21 gets inserted inside the body cavity of the subject and takes images, using the imaging unit 244, of the photographic subject such as the body tissue present at the positions at which the outside light does not reach.

The front end portion 24 includes the following: a light guide 241 that is made of glass fiber and that serves as the light guiding path for the light emitted by the light source unit 3a; an illumination lens 242 that is installed at the front end of the light guide 241; a left-eye optical system 243a for light condensing; a right-eye optical system 243b for light condensing; and the imaging unit 244 that is installed at the image formation positions of the left-eye optical system 243a and the right-eye optical system 243b and that receives the light condensed by the left-eye optical system 243a and the right-eye optical system 243b, performs photoelectric conversion of the received light into electrical signals, and performs predetermined signal processing with respect to the electrical signals.

The left-eye optical system 243a is configured using one or more lenses, is installed at the anterior portion of the imaging unit 244, and forms images using the light coming from the photographic subject. Moreover, the left-eye optical system 243a can be equipped with the optical zoom function for varying the angle of view and with the focusing function for varying the focal point.

The right-eye optical system 243b is made of one or more lenses, is installed in the anterior portion of the imaging unit 244, and forms images using the light coming from the photographic subject. A photographic subject image formed by the right-eye optical system 243b has a parallax with a photographic subject image formed by the left-eye optical system 243a. Moreover, the right-eye optical system 243b can be equipped with the optical zoom function for varying the angle of view and with the focusing function for varying the focal point.

The imaging unit 244 includes a left-eye imaging element 244a and a right-eye imaging element 244b.

The left-eye imaging element 244a receives a drive signal from the processor 3, accordingly performs photoelectric conversion of the light received from the left-eye optical system 243a, and generates electrical signals equivalent to a single frame constituting a single image (i.e., generates left-eye RAW data). More particularly, the left-eye imaging element 244a has a plurality of pixels arranged in a matrix-like manner, where each pixel has a photodiode for storing electrical charge corresponding to the amount of light and has a capacitor for converting the electrical charge transferred from the photodiode into a voltage level. Thus, each pixel performs photoelectric conversion of the light coming from the left-eye optical system 243a and generates an electrical signal. Then, the electrical signals that are generated by such pixels which are arbitrarily set as the targets for reading from among a plurality of pixels are sequentially read and are output as image signals representing RAW data. On the light receiving surface of the left-eye imaging element 244a, for example, a color filter is installed so that each pixel receives light having either one wavelength band from among the wavelength bands of the color components of red (R), green (G), and blue (B).

The right-eye imaging element 244b receives a drive signal from the processor 3, accordingly performs photoelectric conversion of the light received from the right-eye optical system 243b, and generates electrical signals equivalent to a single frame constituting a single image (i.e., generates right-eye RAW data). More particularly, the right-eye imaging element 244b has a plurality of pixels arranged in a matrix-like manner, where each pixel has a photodiode for storing electrical charge corresponding to the amount of light and has a capacitor for converting the electrical charge transferred from the photodiode into a voltage level. Thus, each pixel performs photoelectric conversion of the light coming from the right-eye optical system 243b and generates an electrical signal. Then, the electrical signals that are generated by such pixels which are arbitrarily set as the targets for reading from among a plurality of pixels are sequentially read and are output as image signals representing RAW data. On the light receiving surface of the right-eye imaging element 244b, for example, a color filter is installed so that each pixel receives light having either one wavelength band from among the wavelength bands of the color components of red (R), green (G), and blue (B).

The left-eye imaging element 244a and the right-eye imaging element 244b are implemented using, for example, CCD image sensors (CCD stands for Charge Coupled Device) or CMOS image sensors (CMOS stands for Complementary Metal Oxide Semiconductor). Moreover, the left-eye imaging element 244a as well as the right-eye imaging element 244b can be configured using a single-plate image sensor or using a plurality of image sensors of, for example, the three-plate type.

A left-eye image obtained by the left-eye imaging element 244a and a right-eye image obtained by the right-eye imaging element 244b are images capturing the same photographic subject but having different fields of view and having a parallax therebetween.

Herein, in the present embodiment, it is assumed that the imaging unit 244 includes two imaging elements corresponding to the left-eye optical system 243a and the right-eye optical system 243b that generate electrical signals equivalent to a single frame constituting a single image. Moreover, in the present embodiment, it is assumed that the light obtained as a result of image formation performed by the left-eye optical system 243a and by the right-eye optical system 243b is received by two imaging elements corresponding to the left-eye optical system 243a and the right-eye optical system 243b. Alternatively, the light can be received using a single imaging element by partitioning the light receiving area.

The operating unit 22 includes the following: a curved knob 221 that is meant for bending the curved portion 25 in the vertical direction and the horizontal direction; a treatment tool insertion portion 222 from which a treatment tool such as an electrocautery or a testing probe is insertable; and a plurality of switches 223 representing operation input units that enable input of operation instruction signals regarding the screen display control attributed to the processor 3, an insufflation unit, a water supply unit, or freeze processing. The treatment tool that is inserted from the treatment tool insertion portion 222 passes through a treatment tool channel (not illustrated) of the front end portion 24 and appears from an opening (not illustrated) of the front end portion 24. To each of the switches 223, such as a switch for inputting an instruction to set a measurement mode (described later) and a switch meant for inputting a freeze instruction, an instruction signal is assigned that is output as a result of pressing that switch.

Moreover, the endoscope 2 includes a memory 224 for recording the information of the endoscope 2. In the memory 224, identification information such as the type of the endoscope 2, the model number, and the type of the left-eye imaging element and the right-eye imaging element 244b is recorded. Moreover, the memory 224 can also be used to record various parameters, such as a white balance (WB) adjustment parameter and a variability correction value useful at the time of manufacturing the endoscope 2, that are used in image processing with respect to the image data obtained by imaging by the left-eye imaging element 244a and the right-eye imaging element 244b.

The universal cord 23 has at least the light guide 241 and a cable assembly 245 of one or more signal lines built-in. The cable assembly 245 includes a signal line for transmitting image signals, a signal line for transmitting drive signals meant for driving the imaging unit 244, and a signal line for sending and receiving information containing specific information regarding the endoscope 2 (the imaging unit 244). In the present embodiment, it is assumed that electrical signals are transmitted using signal lines. However, alternatively, optical signals can be transmitted, or transmission of signals between the endoscope 2 and the processor 3 can be performed using wireless communication.

At the time of mounting the endoscope 2 onto the processor 3, the abovementioned information about the endoscope 2 is output to the processor 3 as a result of communication processing performed with the processor 3. Alternatively, there are times when connecting pins are provided according to the rules compliant to the information about the endoscope 2; and, at the time of mounting the endoscope 2, based on the connection state of the connecting pin provided in the processor 3 and the connecting pin provided in the endoscope 2, the processor 3 recognizes the establishment of connection with the endoscope 2.

Given below is the explanation of a configuration of the processor 3. The processor 3 includes a signal processing unit 31, a frame memory 32, a mode setting unit 33, an input unit 34, a control unit 35, and a memory unit 36.

The signal processing unit 31 performs signal processing with respect to the left-eye image data (analog), which is output by the left-eye imaging element 244a, and with respect to the right-eye image data (analog), which is output by the right-eye imaging element 244b; and generates display image data to be displayed in the display device 4 and generates recording image data to be recorded in the recording medium 5. The signal processing unit 31 is configured using a general-purpose processor such as a central processing unit (CPU) or using a dedicated processor having various types of arithmetic circuits meant for implementing specific functions, such as an application specific integrated circuit (ASIC) or a field programmable gate array (FPGA) that is a programmable logic device in which the processing details can be rewritten. Regarding the details of the signal processing unit 31, the explanation is given later.

The frame memory 32 is used to store the image data, which is generated by a preprocessing unit 311, equivalent to the number of frames that is set. In the present embodiment, the frame memory 32 is used to store the image data equivalent to a few frames. When new image data is input to the frame memory 32, the oldest image data from among the currently-stored image data is overwritten with the new image data. Thus, the image data equivalent to a few frames is stored while sequentially updating the image data in order of the image data having the latest acquisition timing. The frame memory 32 is configured using a random access memory (RAM), such as a video RAM (VRAM).

From among the switches 223, when the switch meant for mode setting is pressed and accordingly an instruction signal indicating mode setting is input, the mode setting unit 33 changes the mode setting of the signal processing. More particularly, in the present embodiment, the following two modes can be set: a three-dimensional observation mode in which a parallax image generated based on the left-eye image data and the right-eye image data is displayed in the display device 4; and a measurement mode (a recording image selection mode) in which image data measured using the measurement device 6 is selected as the image data to be recorded in the recording medium 5 and the selected image data is recorded in the recording medium 5. Usually, the three-dimensional observation mode is set. When an instruction signal for instructing mode setting is input, the mode setting unit 33 changes the mode to the measurement mode. The mode setting unit 33 is configured using a general-purpose processor such as CPU or using a dedicated processor having various types of arithmetic circuits meant for implementing specific functions, such as an ASIC or an FPGA.

The input unit 34 is implemented using a keyboard, a mouse, switches, and a touch-sensitive panel; and receives input of various signals such as operation instruction signals for instructing operations of the endoscope system 1. Moreover, the input unit 34 can also include the switches provided on the operating unit 22 and a portable terminal such as an external tablet computer.

The control unit 35 performs drive control with respect to the constituent elements including the imaging unit 244 and the light source unit 3a, and performs input-output control of information with respect to the constituent elements. The control unit 35 refers to control information data (for example, the read timings) stored in the memory unit 36 and used in performing imaging control, and sends the control information data as drive signals to the imaging unit 244 via a predetermined signal line included in the cable assembly 245.

Moreover, based on an instruction signal received by the input unit 34, the control unit 35 performs freeze control or release control using a freeze processing unit 314. In the freeze control, control is performed to display the same image as a freeze-frame in the display device 4 for a longer than usual period of time such as the period of time taken for displaying images equivalent to a few frames. In the release control, for example, the control is performed to record, in the recording medium 5, the image data present at the timing of receiving input of the instruction signal. The control unit 35 is configured using a general-purpose processor such as CPU or using a dedicated processor having various types of arithmetic circuits meant for implementing specific functions, such as an ASIC or an FPGA.

The memory unit 36 is used to store various computer programs meant for operating the endoscope system 1 and to store data containing various parameters required in the operations of the endoscope system 1. Moreover, the memory unit 36 is used to store identification information of the processor 3. The identification information contains specific information (ID), the model year, and the specifications information.

Moreover, the memory unit 36 is used to store various computer programs including an image acquisition processing program that is meant for implementing the image acquisition processing method of the processor 3. The various computer programs can be widely circulated by recorded them in a computer-readable recording medium such as a hard disk, a flash memory, a CD-ROM, a DVD-ROM, or a flexible disk. Alternatively, the various programs can be downloaded via a communication network. Herein, the communication network is implemented using, for example, an existing public line network, a local area network (LAN), or a wide area network (WAN), and can be configured as a wired network or a wireless network.

The memory unit 36 having such a configuration is implemented using a read only memory (ROM), in which various computer programs are installed in advance, and using a RAM or a hard disk in which arithmetic parameters and data of various operations are stored.

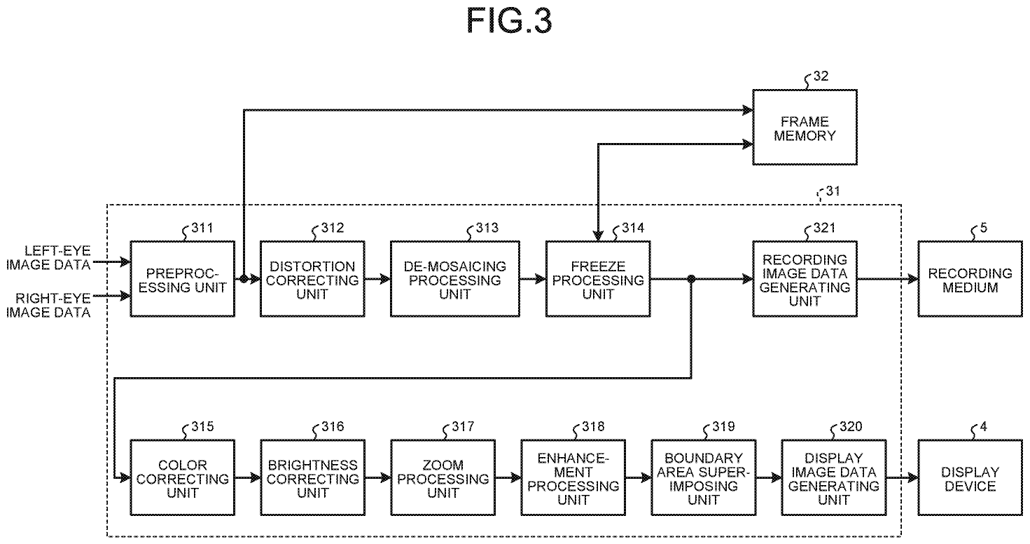

FIG. 3 is a block diagram for explaining a configuration of the signal processing unit of the endoscope system according to the embodiment of the disclosure. The signal processing unit 31 includes the preprocessing unit 311, a distortion correcting unit 312, a de-mosaicing processing unit 313, a freeze processing unit 314, a color correcting unit 315, a brightness correcting unit 316, a zoom processing unit 317, an enhancement processing unit 318, a boundary area superimposing unit 319, a display image data generating unit 320, and a recording image data generating unit 321.

The preprocessing unit 311 performs, with respect to the left-eye image data (analog) output by the left-eye imaging element 244a and the right-eye image data (analog) output by the right-eye imaging element 244b, the following processing: OB clamping for deciding on the black level; white balance correction; gain adjustment; noise removal; A/D conversion; and noise reduction. In the three-dimensional observation mode, the preprocessing unit 311 performs the abovementioned signal processing, and generates left-eye image data (digital) containing a left-eye image having the RGB color components assigned thereto and generates right-eye image data (digital) containing a right-eye image having the RGB color components assigned thereto; and outputs the generated image data to the distortion correcting unit 312 and the frame memory 32. On the other hand, in the measurement mode, the preprocessing unit 311 performs the abovementioned signal processing, and generates left-eye image data (digital) containing a left-eye image having the RGB color components assigned thereto and generates right-eye image data (digital) containing a right-eye image having the RGB color components assigned thereto; outputs either the left-eye image data or the right-eye image data (in the present embodiment, the left-eye image data) to the distortion correcting unit 312; and outputs the left-eye image data and the right-eye image data to the frame memory 32. In the following explanation, the left-eye image data and the right-eye image data is sometimes collectively referred to as image data.

The distortion correcting unit 312 performs distortion correction with respect to the left-eye image data or the right-eye image data input from the preprocessing unit 311. In the distortion correction, for example, distortion aberration is measured using the left-eye image data and the right-eye image data, and accordingly distortion of the left-eye image data as well as the right-eye image data is corrected.

The de-mosaicing processing unit 313 interpolates, in the image data of each color component, the missing pixel values using the surrounding pixel values. As a result, in the image data of each color component, all pixel positions have pixel values (or interpolated values) assigned thereto.

In the three-dimensional observation mode, when input of a freeze instruction signal is received as a result of pressing of one of the switches 223, the freeze processing unit 314 performs freeze-frame display in the display device 4 from the frame memory 32; and selects the image to be recorded in the recording medium 5 and outputs the selected image to the color correcting unit 315. Moreover, in the measurement mode, when input of a release instruction signal is received as a result of pressing of one of the switches 223, the freeze processing unit 314 performs freeze processing for displaying the left-eye image as a freeze-frame, and selects the recording image data corresponding to the left-eye image. More particularly, the freeze processing unit 314 selects the latest left-eye image data, from among the frames stored in the frame memory 32, as the left-eye image data to be freeze-displayed in the display device 4 and outputs that left-eye image data to the color correcting unit 315; and outputs the left-eye image data and the right-eye image data of the latest frame to the recording image data generating unit 321. Meanwhile, when input of a release instruction signal is received, the freeze processing unit 314 can sequentially display the latest left-eye images without performing freeze processing of the left-eye images, and also perform the recording operation.

When neither a freeze instruction signal not a release instruction signal is received, the freeze processing unit 314 outputs predetermined image data in the frame memory 32, such as the image data having the latest acquisition (imaging) timing, to the color correcting unit 315. Meanwhile, after the target image data for freeze processing is selected, within the freeze-frame display period attributed to freeze processing, the freeze processing unit 314 performs only the operation of outputting the image data, which is input from the de-mosaicing processing unit 313, to the frame memory 32. After the freeze processing is terminated, the freeze processing unit 314 selects the latest image data from the frame memory 32 including the image data that is newly input from the de-mosaicing processing unit 313. Hence, the image that is displayed in the display device 4 after the freeze processing is an image that is chronologically discontinuous with the images before the freeze processing on account of losing the latest data within the freeze-frame display period attributed to the freeze processing. As a result, in the images displayed before and after the freeze processing, sometimes the variation in the photographic subject image becomes large as compared to the case of a video display of chronologically continuous images.

In the three-dimensional observation mode, the freeze processing unit 314 selects, as the best frame, an image stored in the frame memory 32, such as the left-eye image having the least blurring from among the left-eye images. The freeze processing unit 314 obtains the left-eye image selected as the best frame and obtains a right-eye image from the frame memory 32, and outputs the images to the color correcting unit 315. More particularly, when input of a freeze instruction signal is received, the freeze processing unit 314 can calculate the blurring in a plurality of left-eye images stored in the frame memory 32 and select the best frame based on the calculated blurring. Alternatively, every time image data is input from the preprocessing unit 311, the freeze processing unit 314 can calculate the blurring of the left-eye image corresponding to the image data, associate the calculated blurring with the frame number of that left-eye image, and store them in the frame memory 32. Herein, the blurring of images is calculated using a known calculation method.

The color correcting unit 315 performs, with respect to the image data input from the freeze processing unit 314, uniform management of the colors that are different among the devices. For example, the color correcting unit 315 performs uniform management of the colors among the endoscope 2, the processor 3, and the display device 4. The color correcting unit 315 is configured using a color management system (CMS).

The brightness correcting unit 316 performs brightness correction with respect to the image data input from the color correcting unit 315. More particularly, the brightness correcting unit 316 uses a y value, which is set in advance, with respect to the left-eye image data and the right-eye image data; and performs gray level correction for brightening the dark portions having low luminance.

The zoom processing unit 317 performs the operation of enlarging the size of the left-eye image or the parallax image according to the enlargement factor set in advance or according to the enlargement factor input via the input unit 34. The enlargement operation performed by the zoom processing unit 317 is possible within the range in which the outer rim of the image fits inside an image display area R₂ (described later). Meanwhile, it is also possible to perform setting to disable enlargement. For example, whether or not to enable or disable the enlargement can be decided using the input unit 34.

The enhancement processing unit 318 performs contour enhancement with respect to the left-eye image data and the right-eye image data that have been subjected to zoom processing by the zoom processing unit 317. As a result, image data in which contours are expressed more clearly gets generated.

In the measurement mode, the boundary area superimposing unit 319 superimposes a frame image, which represents a boundary area, on the left-eye image. The frame image indicates that the measurement accuracy is guaranteed inside the frame. The boundary area superimposing unit 319 uses the frame image stored in advance in the memory unit 36 and the enlargement factor with which the zoom processing unit 317 performed processing; accordingly varies the size of the frame image to be superimposed; and superimposes the post-variation frame image onto the left-eye image. It is desirable that the color of the frame in the frame image is different depending on the wavelength band of the illumination light. For example, when the illumination is attributed to the illumination light of white color including the wavelength bands of blue, green, and red colors, the color of the frame is set to the green color. When the illumination is attributed to the narrow band of blue color and to the narrow-band light made of the narrow band of green color, the color of the frame is set to the red color. In the case of performing illumination using a narrow band, for example, it is desirable from the perspective of frame visibility to set the color of the frame to a complimentary color of the color including the narrow band. At that time, in the measurement mode, the boundary area superimposing unit 319 selects the color of the frame from the wavelength bands of the emitted illumination light, and superimposes the frame image of the selected color onto the left-eye image.

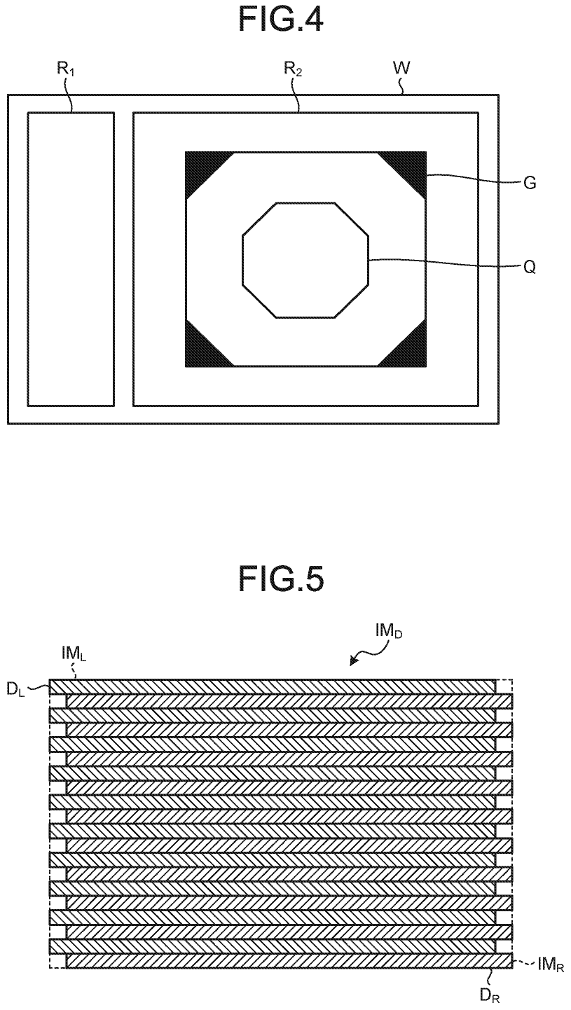

FIG. 4 is a diagram illustrating an exemplary image displayed in the display device of the endoscope system according to the embodiment of the disclosure. In FIG. 4 is illustrated an image in the measurement mode. A display image W in the display device 4 includes an information display area R₁ meant for displaying the information such as the settings about the subject and the endoscope, and includes the image display area R₂ meant for displaying the image generated by the signal processing unit 31. In the image display area R₂, a parallax image is displayed in the three-dimensional observation mode, while an image obtained by superimposing a frame image Q onto a left-eye image G is displayed in the measurement mode.

The user performs adjustment in such a way that the region measured using the measurement device 6 (i.e., the measured region) is positioned inside the frame image Q, and records the post-adjustment image in the recording medium 5. When the measurement device 6 performs measurement of such a recorded image, the measurement is performed without much impact of optical distortion.

In the three-dimensional observation mode, the display image data generating unit 320 generates a parallax image with respect to the image data as a result of causing a parallax by mutually shifting the left-eye image and the right-eye image; performs signal processing in such a way that the signals are in a displayable form in the display device 4; and generates the display image data. More particularly, based on the amount of shift set in advance, the display image data generating unit 320 relatively shifts the right-eye image and the left-eye image, and generates display image signals. In the three-dimensional observation mode, the parallax image generated by the display image data generating unit 320 is expressed in the three-dimensional coordinate system.

Explained below with reference to FIG. 5 is a parallax image generated by the display image data generating unit 320. FIG. 5 is a diagram for explaining a parallax image generated in the endoscope system according to the embodiment of the disclosure. As illustrated in FIG. 5, the display image data generating unit 320 generates a parallax image IM_{D} by arranging line images D_{L} of horizontal lines in a left-eye image IM_{L} and line images D_{R} of horizontal lines in a right-eye image IM_{R} in an alternate manner while shifting them according to the amount of shift set in advance. More particularly, the display image data generating unit 320 arranges the line images D_{L} of the odd lines included in the left-eye image IM_{L} and the line images D_{R} of the even lines included in the right-eye image IM_{R} in an alternate manner while shifting them according to the set amount of shift. Thus, the parallax image IM_{D} can also be called a line-by-line image. Regarding the horizontal lines mentioned herein, in an imaging element in which a plurality of pixels is arranged in a matrix-like manner, the lines formed by the pixels arranged in one of the two arrangement directions correspond to the horizontal lines.

On the other hand, in the measurement mode, with respect to the left-eye image data on which a frame image is superimposed, the display image data generating unit 320 performs signal processing in such a way that the signals are in a displayable form in the display device 4; and generates the display image signals. The left-eye image for display as generated by the display image data generating unit 320 in the measurement mode is a photographic subject image expressed in the two-dimensional coordinate system.

The display image data generating unit 320 places the parallax image data or the left-eye image data in the image display area R₂ and generates display image data to be displayed along with the information display area R₁ in the display device 4 (for example, see FIG. 4). Then, the display image data generating unit 320 sends the image data of the display image to the display device 4.

Meanwhile, when the input image data is the target image data for freeze processing, the display image data generating unit 320 displays the target image as a freeze-frame in the display device 4 for a period of time set in advance, such as a period of time in which images of a few frames are displayed.

The recording image data generating unit 321 generates recording data in which image data (the left-eye image data and the right-eye image data), which is subjected to preprocessing by the preprocessing unit 311 but which is not yet subjected to distortion correction, is held in a corresponding manner to the parameters required in the operations to be performed after distortion correction. For example, the recording image data generating unit 321 records, for example, the post-preprocessing left-eye image data and the post-preprocessing right-eye image data selected by the freeze processing unit 314; records the distortion correction parameter, records the zoom processing parameter; and records the specific information of the endoscope 2 in the recording medium 5. The distortion correction parameter that is recorded by the recording image data generating unit 321 in the recording medium 5 has a greater data volume and enables high-accuracy distortion correction as compared to the distortion correction parameter used by the distortion correcting unit 312. At that time, the parameters and the specific information of the endoscope 2 can be embedded in the header portion of the image data, or can be recorded as a text file that is separate from the image data but that is associated to the image data.

The recording image data generating unit 321 generates a parallax image with respect to the image data, which is selected by the freeze processing unit 314, as a result of causing a parallax by mutually shifting the left-eye image and the right-eye image; performs signal processing in such a way that the signals are in a displayable form in the display device 4; and treats the generated parallax image as the recording image data. Examples of a parallax image include a line-by-line image as explained earlier, a side-by-side image in which the left-eye image and the right-eye image are arranged in the direction of the horizontal lines, and a top-and-bottom image in which the left-eye image and the right-eye image are arranged in the direction of the vertical lines. At that time, the parameters and the specific information of the endoscope 2 can be embedded in the header portion of the parallax image of the line-by-line type, or can be recorded as a text file that is separate from the image data but that is associated to the image data.

Given below is the explanation of a configuration of the light source unit 3a. The light source unit 3a includes an illumination unit 301 and an illumination control unit 302. Under the control of the illumination control unit 302, the illumination unit 301 sequentially switches among illumination light of different exposure amounts and emits illumination light onto the photographic subject (the subject). The illumination unit 301 includes a light source 301a and a light source driver 301b.

The light source 301a is configured using a light source that emits white light or using one or more lenses, and emits light (illumination light) as a result of driving of an LED light source. The illumination light generated by the light source 301a is emitted toward the photographic subject from the front end of the front end portion 24 via the light guide 241. In the present embodiment, it is assumed that white light is emitted. Alternatively, in order to perform NBI observation, the light source 301a can be configured to emit, as illumination light, narrow-band light made of the light of the narrow band of blue color (for example, 390 nm to 445 nm) and the light of the narrow band of green color (for example, 530 nm to 550 nm); or can be configured to able to switch between emitting white light and emitting narrow-band light. The light source 301a is implemented using an LED light source, or a laser light source, or a xenon lamp, or a halogen lamp.

Under the control of the illumination control unit 302, the light source driver 301b supplies electrical power to the light source 301a and makes the light source 301a emit illumination light.

Based on the control signals (light control signals) received from the control unit 35, the illumination control unit 302 controls the electrical energy to be supplied to the light source 301a and controls the drive timing of the light source 301a.

The display device 4 displays display images corresponding to the image signals received from the processor 3 (the display image data generating unit 320) via a video cable. The display device 4 is configured using a liquid crystal monitor or an organic electroluminescence (organic EL) monitor.

The measurement device 6 reads data from the recording medium 5 and obtains the data generated by the recording image data generating unit 321; generates a parallax image from the obtained image data and parameters; and displays a parallax image. The measurement device 6 calculates, regarding a position specified in the parallax image, the distance from the endoscope 2 to the photographic subject. The user uses the input unit 34 and instructs a position for measurement in the displayed image. In response, the measurement device 6 measures the distance of the measure point. As the measurement method, a known method such as stereo measurement (for example, triangulation) can be implemented.

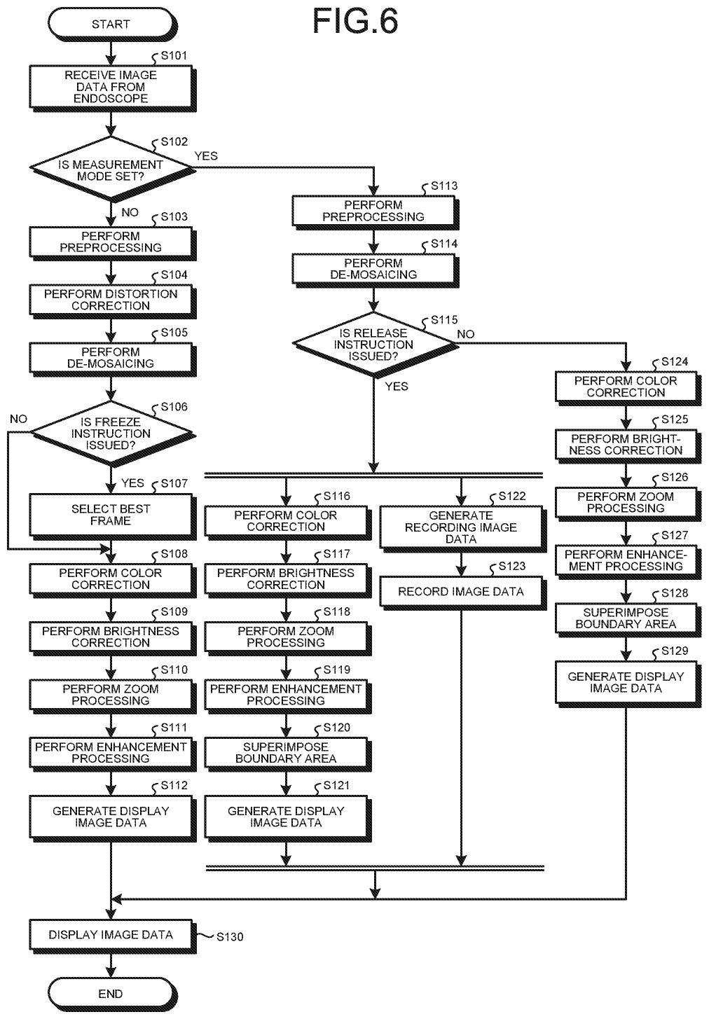

Given below is the explanation of the image processing performed in the endoscope system 1. FIG. 6 is a flowchart for explaining the image processing performed in the endoscope system according to the embodiment of the disclosure. In the following explanation, it is assumed that the constituent elements perform operations under the control of the control unit 35.

Firstly, the processor 3 receives image data from the endoscope 2 (Step S101). The control unit 35 determines whether or not the measurement mode is set (Step S102). If the measurement mode is not set, that is, if the three-dimensional observation mode is set (No at Step S102), then the system control proceeds to Step S103. On the other hand, if the measurement mode is set (Yes at Step S102); then the system control proceeds to Step S113.

At Step S103, the preprocessing unit 311 performs the preprocessing with respect to the received image data (the left-eye image data and the right-eye image data), and outputs the post-preprocessing image data to the distortion correcting unit 312 and the frame memory 32.

At Step S104 that follows Step S103, the distortion correcting unit 312 performs distortion correction with respect to the left-eye image data and the right-eye image data input from the preprocessing unit 311. The distortion correcting unit 312 corrects the distortion for each color component of the left-eye image data and the right-eye image data. Then, the distortion correcting unit 312 outputs the post-distortion-correction left-eye image data and the post-distortion-correction right-eye image data to the de-mosaicing processing unit 313.

At Step S105 that follows Step S104, the de-mosaicing processing unit 313 interpolates, in the image data of each color component, the missing pixel values using the surrounding pixel values. Then, the de-mosaicing processing unit 313 outputs the post-interpolation image data to the color correcting unit 315 via the freeze processing unit 314.

At Step S106 that follows Step S105, the freeze processing unit 314 determines whether or not a freeze instruction is input as a result of pressing of one of the switches 223. If it is determined that a freeze instruction is input (Yes at Step S106), then the system control proceeds to Step S107. On the other hand, if it is determined that a freeze instruction is not input (No at Step S106), then the freeze processing unit 314 outputs the image data (the left-eye image data and the right-eye image data) of the latest frame stored in the frame memory 32 to the color correcting unit 315, and the system control proceeds to Step S108.

At Step S107, the freeze processing unit 314 selects, as the best frame, an image stored in the frame memory 32, such as the left-eye image having the least blurring from among the left-eye images. Then, the freeze processing unit 314 outputs the image data (the left-eye image data and the right-eye image data) of the selected best frame to the color correcting unit 315.

At Step S108, the color correcting unit 315 performs, with respect to the image data input from the freeze processing unit 314, uniform management of the colors that are different among the devices. For example, the color correcting unit 315 converts the color space of the image data according to the color space of the display device 4. Then, the color correcting unit 315 outputs the post-conversion image data to the brightness correcting unit 316.

At Step S109 that follows Step S108, the brightness correcting unit 316 performs brightness correction with respect to the left-eye image data and the right-eye image data. Then, the brightness correcting unit 316 outputs the post-brightness-correction image data to the zoom processing unit 317.

At Step S110 that follows Step S109, the zoom processing unit 317 enlarges the size of the left-eye image data and the right-eye image data according to the enlargement factor set in advance or according to the enlargement factor input using the input unit 34. Then, the zoom processing unit 317 outputs the post-zoom-processing image data to the enhancement processing unit 318.

At Step S111 that follows Step S110, the enhancement processing unit 318 performs contour enhancement with respect to the left-eye image data and the right-eye image data that have been subjected to zoom processing by the zoom processing unit 317. Then, the enhancement processing unit 318 outputs the post-enhancement-processing image data to the display image data generating unit 320 via the boundary area superimposing unit 319.

At Step S112 that follows Step S111, the display image data generating unit 320 generates a parallax image with respect to the image data as a result of causing a parallax by mutually shifting the left-eye image and the right-eye image; performs signal processing in such a way that the signals are in a displayable form in the display device 4; and generates display image data. Once the display image data is generated, the system control proceeds to Step S130.

Meanwhile, at Step S113, the preprocessing unit 311 performs preprocessing with respect to the received image data (the left-eye image data and the right-eye image data); outputs the post-preprocessing left-eye image data to the de-mosaicing processing unit 313 via the distortion correcting unit 312; and outputs the post-preprocessing left-eye image data and the post-preprocessing right-eye image data to the frame memory 32. At that time, the image data output to the de-mosaicing processing unit 313 and the frame memory 32 is image data not subjected to distortion correction.

At Step S114 that follows Step S113, the de-mosaicing processing unit 313 interpolates, in the image data of each color component of the left-eye image data, the missing pixel values using the surrounding pixel values. Then, the de-mosaicing processing unit 313 outputs the post-interpolation left-eye image data to the freeze processing unit 314.

At Step S115 that follows Step S114, the freeze processing unit 314 determines whether or not a release instruction is input as a result of pressing of one of the switches 223. If it is determined that a release instruction is input (Yes at Step S115), then the freeze processing unit 314 outputs the left-eye image data of the latest frame stored in the frame memory 32 to the color correcting unit 315 and outputs the image data (the left-eye image data and the right-eye image data) of the latest frame to the recording image data generating unit 321. Then, the system control proceeds to Step S116 and Step S122. On the other hand, if it is determined that a release instruction is not input (No at Step S115), then the freeze processing unit 314 outputs the left-eye image data of the latest frame to the color correcting unit 315. Then, the system control proceeds to Step S124.

At Step S116 that follows Step S115, the color correcting unit 315 performs, with respect to the left-eye image data input from the freeze processing unit 314, uniform management of the colors that are different among the devices. Then, the color correcting unit 315 outputs the post-conversion image data to the brightness correcting unit 316.

At Step S117 that follows Step S116, the brightness correcting unit 316 performs brightness correction with respect to the left-eye image data. Then, the brightness correcting unit 316 outputs the post-brightness-correction left-eye image data to the zoom processing unit 317.

At Step S118 that follows Step S117, the zoom processing unit 317 enlarges the size of the left-eye image data according to the enlargement factor set in advance or according to the enlargement factor input using the input unit 34. Then, the zoom processing unit 317 outputs the post-zoom-processing left-eye image data to the enhancement processing unit 318.

At Step S119 that follows Step S118, the enhancement processing unit 318 performs contour enhancement with respect to the left-eye image data that has been subjected to zoom processing by the zoom processing unit 317. Then, the enhancement processing unit 318 outputs the post-enhancement-processing left-eye image data to the boundary area superimposing unit 319.

At Step S120 that follows Step S119, the boundary area superimposing unit 319 superimposes a frame image, which represents the boundary area, on the left-eye image (for example, see FIG. 4). The boundary area superimposing unit 319 uses the frame image stored in advance in the memory unit 36 and the enlargement factor with which the zoom processing unit 317 performed processing; accordingly varies the size of the frame image to be superimposed; and superimposes the post-variation frame image onto the left-eye image. At that time, if a plurality of patterns of the wavelength bands of the illumination light is available, then the boundary area superimposing unit 319 selects the color of the frame from the wavelength band of the emitted illumination light and superimposes the frame image of the selected color onto the left-eye image. Then, the boundary area superimposing unit 319 outputs the post-superimposing superimposed image data to the display image data generating unit 320.

At Step S121 that follows Step S120, the display image data generating unit 320 performs signal processing with respect to the superimposed image data in such a way that the signals are in a displayable form in the display device 4; and generates display image data. Once the display image data is generated, the system control proceeds to Step S130.

Meanwhile, in parallel to the operations performed at Step S116 to Step S121, the recording image data generating unit 321 generates recording image data to be recorded in the recording medium 5 (Step S122). The recording image data generating unit 321 generates recording data in which the left-eye image data and the right-eye image data of the latest frame as selected by the freeze processing unit 314 is held in a corresponding manner to the parameters required in the operations to be performed after distortion correction.

At Step S123 that follows Step S122, the recording image data generating unit 321 records the recording data, which is generated at Step S121, in the recording medium 5. Once the recording data is recorded in the recording medium, the system control proceeds to Step S130.

Meanwhile, at Step S124, the color correcting unit 315 performs, with respect to the left-eye image data input from the freeze processing unit 314, uniform management of the colors that are different among the devices. Then, the color correcting unit 315 outputs the post-conversion left-eye image data to the brightness correcting unit 316.

At Step S125 that follows Step S124, the brightness correcting unit 316 performs brightness correction with respect to the left-eye image data. Then, the brightness correcting unit 316 outputs the post-brightness-correction left-eye image data to the zoom processing unit 317.

At Step S126 that follows Step S125, the zoom processing unit 317 enlarges the size of the left-eye image data according to the enlargement factor set in advance or according to the enlargement factor input using the input unit 34. Then, the zoom processing unit 317 outputs the post-zoom-processing left-eye image data to the enhancement processing unit 318.

At Step S127 that follows Step S126, the enhancement processing unit 318 performs contour enhancement with respect to the left-eye image data and the right-eye image data that have been subjected to zoom processing by the zoom processing unit 317. Then, the enhancement processing unit 318 outputs the post-enhancement-processing left-eye image data to the boundary area superimposing unit 319.

At Step S128 that follows Step S127, the boundary area superimposing unit 319 superimposes a frame image, which represents the boundary area, onto the left-eye image (for example, see FIG. 4). The boundary area superimposing unit 319 uses the frame image stored in advance in the memory unit 36 and the enlargement factor with which the zoom processing unit 317 performed processing; accordingly varies the size of the frame image to be superimposed; and superimposes the post-variation frame image onto the left-eye image. Then, the boundary area superimposing unit 319 outputs the post-superimposing superimposed image data to the display image data generating unit 320.

At Step S129 that follows Step S128, the display image data generating unit 320 performs signal processing with respect to the superimposed image data in such a way that the signals are in a displayable form in the display device 4; and generates display image data. Once the display image data is generated, the system control proceeds to Step S130.

At Step S130, under the control of the control unit 35, the display image data generating unit 320 displays, in the display device 4, the display image including the parallax image generated at Step S112, or the display image including the left-eye image generated at Step S121, or the display image including the left-eye image generated at Step S129.

Meanwhile, the display operation performed at Step S130 can be performed before the operation performed at Step S123, or can be performed at the same time at which the operation at Step S123 is performed.

According to the embodiment of the disclosure, in the measurement mode, a frame image is superimposed on the image obtained by the endoscope 2, and then the superimposed image is displayed. Hence, the user becomes able to perform such adjustment that the measured position lies inside the frame image, and becomes able to record the post-adjustment image in the recording medium 5. As a result, at the time of performing measurement using the measurement device 6, the measurement is performed in the state in which the measure point is positioned within the area in which the measurement accuracy is guaranteed. As a result, it becomes possible to hold down a decline in the measurement accuracy.

Moreover, according to the present embodiment, in the measurement mode, the frame image is superimposed after the enhancement processing. However, for example, if the frame image is superimposed before the enhancement processing, the post-superimposition enhancement processing sometimes leads to the enhancement of the frame image thereby resulting in an unnatural image. According to the present embodiment, a display image can be generated in which the superimposed frame image is not unnaturally enhanced. Meanwhile, as long as the visibility can be secured even after enhancing the frame image, then the frame image can be superimposed before the enhancement processing.

Furthermore, according to the present embodiment, in the measurement mode, at the time of generating a two-dimensional display image based on a left-eye image, distortion correction is not performed. As a result, in the measurement mode, it becomes possible to reduce the sensation of fatigue of the user at the time of observing the two-dimensional image.

Moreover, in the present embodiment, the amount of illumination light in the measurement mode can be set to be smaller than the amount of illumination light in the three-dimensional observation mode. At Step S102 in the flowchart explained above, if it is determined that the measurement mode is set (Yes at Step S102), then the control unit 35 performs control to reduce the amount of illumination light at the time when the system control proceeds to Step S113. Alternatively, instead of reducing the amount of illumination light, the brightness correction parameter can be set to have a smaller output value than the brightness correction parameter used at the time of generating three-dimensional image data. As a result of lowering the brightness of the images in the measurement mode than the brightness in the three-dimensional observation mode, it becomes possible to hold down the halation of the measurement target thereby enabling measurement in a more reliable manner.

Furthermore, in the present embodiment, the freeze processing unit 314 selects the best frame having the least blurring in the three-dimensional observation mode; and selects the latest frame in the measurement mode. However, that is not the only possible case. Alternatively, for example, the freeze processing unit 314 can select the latest frame in the third-dimensional observation mode, and can select the best frame in the measurement mode.

Moreover, in the present embodiment, the recording image data generating unit 321 associates the image data with the parameters for distortion correction and the like, and the specific information of the endoscope 2, and records them in the recording medium 5. However, alternatively, data not containing the specific information of the endoscope 2 can be treated as the recording data. Meanwhile, if the recording data contains the specific information of the endoscope 2, then the measurement device 6 becomes able to perform the measurement operation by also taking into account the variability specific to the endoscope 2.

Furthermore, in the present embodiment, the parallax image is a line-by-line image. However, that is not the only possible case. Alternatively, for example, an image having a parallax can be a side-by-side image in which the left-eye image IM_{L} and the right-eye image IM_{R} are arranged in the direction of the horizontal lines, or can be a top-and-bottom image in which the left-eye image IM_{L} and the right-eye image IM_{R} are arranged in the direction of the vertical lines. Moreover, instead of having only a single parallax image, for example, as in the case of the frame sequential method, the left-eye images and the right-eye images can be alternately output and recorded in the recording medium 5.

Furthermore, in the present embodiment, the explanation is given under the premise that a simultaneous illumination/imaging system is implemented in which the imaging unit 244 receives the reflected light of the illumination light. Alternatively, it is possible to implement a sequential illumination/imaging system in which the light source unit 3a individually and sequentially emits the light of the wavelength band of each color component and the imaging unit 244 receives the light of each color component.

Moreover, in the present embodiment, the light source unit 3a is configured as a separate entity from the endoscope 2. Alternatively, for example, a light source device can be installed inside the endoscope 2, such as a semiconductor light source can be installed at the front end of the endoscope 2. Furthermore, the endoscope 2 can be equipped with the functions of the processor 3.

Moreover, in the present embodiment, the light source unit 3a is integrated with the processor 3. However, alternatively, the light source unit 3a and the processor 3 can be separate devices and, for example, the illumination unit 301 and the illumination control unit 302 can be installed on the outside of the processor 3. Furthermore, the light source 301a can be installed at the front end of the front end portion 24.

Moreover, in the present embodiment, as the endoscope system according to the disclosure, the endoscope system 1 is used that includes the flexible endoscope 2 for treating the body tissue inside the subject as the observation target. However, alternatively, it is also possible to use an endoscope system in which a rigid endoscope is used, or an industrial endoscope is used for observing the characteristics of materials, or a capsule endoscope is used, or a fiberscope is used, or a device is used having a camera head connected to the eyepiece of an optical endoscope such as an optical visual tube.

Furthermore, in the present embodiment, the explanation is given with reference to an endoscope system. Alternatively, for example, the present embodiment can be implemented also in the case of outputting videos to an electronic view finder (EVF) installed in a digital still camera.

As described above, the image processing device, the image processing system, and the image processing method according to the disclosure are useful in holding down a decline in the measurement accuracy.

Thus, according to the disclosure, it becomes possible to hold down a decline in the measurement accuracy.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing device configured to obtain first-type image data and second-type image data generated as a result of taking images of same photographic subject from different directions, and generate a parallax image based on the first-type image data and the second-type image data, the image processing device comprising:
a processor comprising hardware, wherein the processor is configured to:
set an image selection mode in which an image is selectable based on an image displayed in a display;
superimpose a frame data onto image data to be displayed in the display when the image selection mode is set, the frame data representing a boundary area whose size is set based on an optical system that forms an image of the photographic subject; and
obtain the first-type image data and the second-type image data corresponding to selected image when the image selection mode is set.

2. The image processing device according to claim 1, wherein
the image selection mode is used to display a two-dimensional image that is generated based on the first-type image data, in the display, and
the processor is configured to superimpose the frame image onto the first-type image data.

3. The image processing device according to claim 2, wherein the processor is configured to
either set a three-dimensional observation mode in which a parallax image that is generated based on the first-type image data and the second-type image data is displayed in the display,
or set the image selection mode.

4. The image processing device according to claim 1, wherein the processor is configured to vary color of the frame image according to wavelength band of illumination light, and superimpose the frame image onto image data to be displayed in the display.

5. The image processing device according to claim 1, wherein
the processor is further configured to enlarge image that is to be displayed in the display, according to enlargement factor that is set, and
the processor is configured to enlarge the frame image based on the enlargement factor, and superimpose the frame image onto image data to be displayed in the display.

6. The image processing device according to claim 3, wherein the processor is further configured to, in the three-dimensional observation mode, correct image distortion that is attributed to the optical system, in the parallax image.

7. The image processing device according to claim 6, wherein
the processor is further configured to, when the image selection mode is set, generate recording image data to be recorded in a recorded medium, and
the processor is further configured to generate the recording image data in which parameter having a greater data volume than parameter used when the image distortion of the parallax image is corrected is associated with the image data.

8. The image processing device according to claim 3, further comprising a controller configured to set brightness of a two-dimensional image that is generated in the image selection mode, to be different than brightness of the parallax image that is generated in the three-dimensional observation mode.

9. The image processing device according to claim 1, wherein the image processing device is an endoscope image processing device configured to generate the parallax image based on the first-type image data and the second-type image data generated as a result of images taken by an endoscope that is inserted inside a subject.

10. An image processing system comprising:
an imaging unit including an optical system configured to form a photographic subject image and an image sensor configured to receive light obtained as a result of image formation performed by the optical system and perform photoelectric conversion, the imaging unit being configured to generate first-type image data and second-type image data generated as a result of taking images of same photographic subject from different directions; and
a processor comprising hardware, wherein the processor is configured to
set an image selection mode in which an image is selectable based on an image displayed in a display,
superimpose a frame data onto image data to be displayed in the display when the image selection mode is set, the frame data representing a boundary area whose size is set based on the optical system that forms an image of the photographic subject, and
obtain the first-type image data and the second-type image data corresponding to selected image when the image selection mode is set.

11. An image processing method of obtaining first-type image data and second-type image data generated as a result of taking images of same photographic subject from different directions, and generating a parallax image based on the first-type image data and the second-type image data, the image processing method comprising:
superimpose a frame data onto image data to be displayed in a display when observation mode is set to an image selection mode in which an image is selectable based on an image displayed in the display, the frame data representing a boundary area whose size is set based on an optical system that forms an image of the photographic subject; and
obtaining the first-type image data and the second-type image data corresponding to selected image when the observation mode is set to the image selection mode.
